(19) United States
(12) Patent Application Publication
(10) Pub. No.: US 2021/0187265 A1
(43) Pub. Date: Jun. 24, 2021

(54) COUPLING DEVICE

(71) Applicant: EZ-E-Fil, LLC, Sonoma, CA (US)

(72) Inventor: Matthew SCHULER, Chico, CA (US)

(21) Appl. No.: 17/131,259

(22) Filed: Dec. 22, 2020

Related U.S. Application Data
(60) Provisional application No. 62/952,952, filed on Dec. 23, 2019.

(51) Int. Cl. A61M 39/10

(52) U.S. Cl. CPC A61M 39/10; A61M 2039/1038; A61M 2039/1077

(57) ABSTRACT

A coupling device includes a female fitting adapted to attach a mixing jar thereto, and a male fitting adapted to attach a vaginal applicator or an oral syringe thereto, or a male fitting adapted to attach a Luer-Lock syringe or a Slip-Tip syringe thereto. Further, a method for coupling a mixing jar to a vaginal applicator or an oral syringe includes providing a coupling device having a female fitting adapted to attach the mixing jar, and a male fitting adapted to attach the vaginal applicator or the oral syringe thereto. The female fitting of the coupling device is then attached to the mixing jar, and the male fitting of the coupling device is attached to the vaginal applicator or the oral syringe. Still further, a method for coupling a mixing jar to a Luer-Lock syringe or a Slip-Tip syringe includes providing a coupling device having a female fitting adapted to attach the mixing jar, and a male fitting adapted to attach the Luer-Lock syringe or the Slip-Tip syringe thereto. The female fitting of the coupling device is then attached to the mixing jar and the male fitting of the coupling device is attached to the Luer-Lock syringe or the Slip-Tip syringe.

### CROSS-REFERENCE TO RELATED APPLICATION

This application claims the benefit of U.S. Provisional Patent Application No. 62/952,952, filed Dec. 23, 2019, which is incorporated by reference herein in its entirety.

### FIELD

The present disclosure relates to coupling devices. More particularly, the present disclosure relates to coupling devices and related methods for coupling mixing jars to vaginal applicators, oral syringes, Luer-Lock syringes, and/or Slip-Tip syringes to facilitate compounding pharmacists and/or patients in the measurement and dosing of certain custom-mixed topical/vaginal creams, which treat a variety of needs such as hormone replacement for both men and women, vaginal dryness, vaginal yeast infections, and other indications.

### BACKGROUND

The traditional role of compounding pharmacies is to make drugs prescribed by doctors for specific patients with needs that can't be met by commercially available drugs. Examples include the following: 1) a patient may need a small or highly accurate dose of a drug that is only available in one standard concentration or 2) a patient may be allergic to one of the ingredients in a commercial version of a drug which requires a custom compound to address the patient's needs.

Topical/vaginal creams are almost universally prepared by compounding pharmacists by using mixing equipment, Unguator EMP (Electronic Mortar & Pestal), manufactured and sold by Gako International GmbH. This mixing equipment provides different size mixing jars (EMP Jars), which can be chosen by the pharmacist depending on the quantity of the compound desired.

Once the topical cream or vaginal cream is mixed, it must be provided to the patient(s) in such a way that it is easy to dose and administer and ideally requires the least amount of cost and time invested by the compounding pharmacist. A number of methods are currently used to accomplish this, none of which is entirely satisfactory.

Accordingly, a new method is needed that eliminates or greatly reduces the disadvantages created by the current methods.

### SUMMARY

Disclosed herein are coupling devices and related methods for facilitating the delivery and dosing of certain compounded topical/vaginal creams for compounding pharmacists and/or their patient customers. As an example, a common application would be custom hormone replacement therapy regimes that are not available in commercial tablet form, where the patient may need a small, custom, liquid dose of a drug mixture, either topically applied to the skin or delivered via a vaginal applicator.

In some embodiments, the coupling device comprises: a female fitting adapted attach a mixing jar thereto; and a male fitting adapted to attach a vaginal applicator or an oral syringe thereto; or a male fitting adapted to attach a Luer-Lock syringe or a Slip-Tip syringe thereto.

In some embodiments, the method for coupling a mixing jar to a vaginal applicator or an oral syringe comprises: providing a coupling device comprising: a female fitting adapted to attach the mixing jar; and a male fitting adapted to attach the vaginal applicator or the oral syringe thereto; attaching the female fitting of the coupling device to the mixing jar; and attaching the male fitting of the coupling device to the vaginal applicator or the oral syringe.

In some embodiments, the method for coupling a mixing jar to a Luer-Lock syringe or a Slip-Tip syringe comprises: providing a coupling device comprising a female fitting adapted to attach the mixing jar; and a male fitting adapted to attach the Luer-Lock syringe or the Slip-Tip syringe thereto; attaching the female fitting of the coupling device to the mixing jar; and attaching the male fitting of the coupling device to the Luer-Lock syringe or the Slip-Tip syringe.

In some embodiments, the female fitting includes a top wall and a cylindrical wall depending from a peripheral edge of the top wall.

In some embodiments, the cylindrical wall includes an inner surface and a thread disposed on the inner surface.

In some embodiments, the male fitting includes a cylindrical member disposed on the top wall of the female fitting.

In some embodiments, the cylindrical member has an outer surface, an inner surface, a thread disposed on the outer surface and the inner surface defining a passageway.

In some embodiments, the thread is a screw-thread or a Luer-Lock thread.

In some embodiments, the thread is a screw-thread and the inner surface is tapered.

### BRIEF DESCRIPTION OF THE DRAWING

The disclosure is best understood from the following detailed description when read in conjunction with the accompanying drawing. It is emphasized that, according to common practice, the various features of the drawing are not necessarily to scale. On the contrary, the dimensions of the various features may be arbitrarily expanded or reduced for clarity. Like numerals denote like features throughout the specification and the drawing.

FIG. 1A is a perspective view of a first embodiment of a coupling device;

FIG. 1B is a side view of the coupling device of FIG. 1A;

FIG. 1C is a top view of the coupling device of FIG. 1A;

FIG. 1D is a cross-sectional view of the coupling device of FIG. 1A through line 1D-1D of FIG. 1C;

FIG. 2A is a perspective view of a second embodiment of a coupling device;

FIG. 2B is a side view of the coupling device of FIG. 2A;

FIG. 2C is a top view of the coupling device of FIG. 2A;

FIG. 2D is a cross-sectional view of the coupling device of FIG. 2A through line 2D-2D of FIG. 2C;

FIG. 3 is perspective view of an embodiment of a mixing jar;

FIG. 4A is a plan view that shows the coupling device of FIGS. 1A-1D coupling a vaginal applicator with the mixing jar of FIG. 3;

FIG. 4B is a plan view that shows the coupling device of FIGS. 1A-1D coupling an oral syringe with the mixing jar of FIG. 3;

FIG. 5A is a plan view that shows the coupling device of FIGS. 2A-2D coupling a Luer-Lock syringe with the mixing jar of FIG. 3; and

FIG. 5B is a plan view that shows the coupling device of FIGS. 2A-2D coupling a Slip-Tip syringe with the mixing jar of FIG. 3.

### DETAILED DESCRIPTION

It should be understood that the phraseology and terminology used below for the purpose of description and should not be regarded as limiting. The use herein of the terms “comprising,” “including,” “having,” “containing,” and variations thereof are meant to encompass the structures and features recited thereafter and equivalents thereof as well as additional structures and features. Unless specified or limited otherwise, the terms “attached,” “mounted,” “affixed,” “connected,” “supported,” “coupled,” and variations thereof are used broadly and encompass both direct and indirect forms of the same.

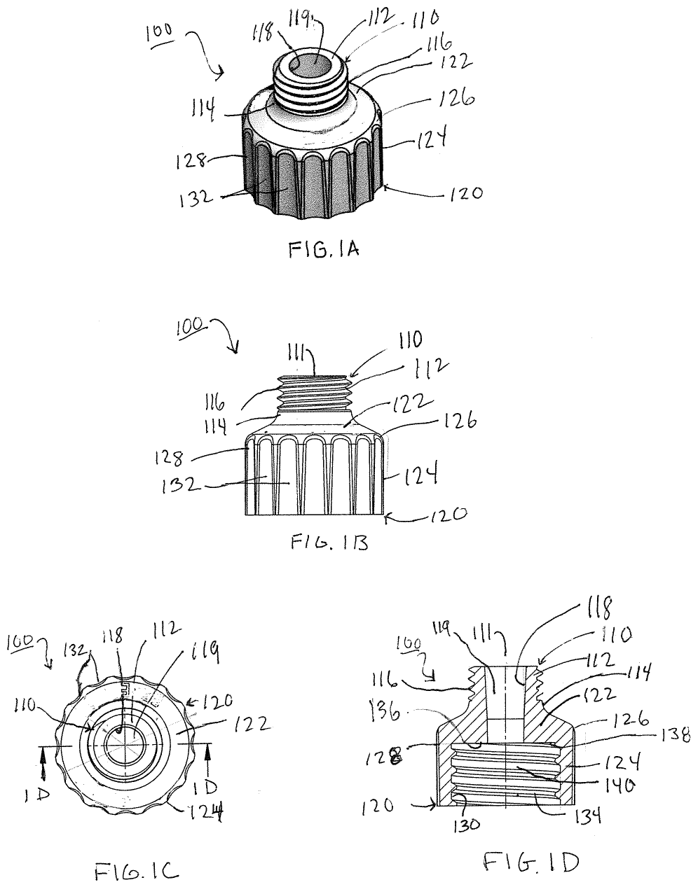

Referring now to FIGS. 1A-1D, there is shown a first embodiment of a coupling device 100 for coupling a mixing jar (e.g., an Electronic Mortar and Pestal (EMP) jar) to a vaginal applicator or an oral syringe. The coupling device 100 generally comprises a male fitting 110 and a female fitting 120.

The female fitting 120 of the coupling device 100 comprises an upwardly tapered circular top wall 122 and a cylindrical side wall 124 depending from a rounded peripheral edge 126 of the top wall 124. The cylindrical side wall 124 of the female fitting 120 has an outer surface 128 and an inner surface 130. The outer surface 128 of the side wall 124 includes a plurality of longitudinal scallops or grooves 132, which extend into the rounded peripheral edge 126 of the top wall 124 and aid in gripping the coupling device 100 when attaching a vaginal applicator or an oral syringe to the coupling device 100 and/or when connecting the coupling device 100 to a mixing jar 150. A standard female or internal thread 134 is provided on the inner surface 130 of the cylindrical side wall 124.

As shown FIGS. 1D, 3, 4A and 4B, the female or internal thread 134 of the female fitting 120 is adapted to complement a male thread 158 provided on a male fitting 156 extending up from a top wall 154 of a lid 152 of the mixing jar 150. The internally threaded female fitting 120 allows the coupling device 100 to be screw-threaded onto the lid 152 of the mixing jar 150. An inner surface 136 of the top wall 122 of the female fitting 120 defines a shoulder 138 above the internal thread 134 which is operative as a stop to ensure exact screw-tightening height and to prevent overtightening of the female fitting 120 of the coupling device 100 to the male fitting 156 of the mixing jar lid 152. More particularly, a free end 160 of the male fitting 156 of the mixing jar lid 152 will abut against the shoulder 138 of the top wall 122 of the female fitting 120 when the coupling device 100 is properly screw-tightened to the male fitting 156 of the mixing jar lid 152. The stop 138 is also operative as a seal to prevent leakage during product transfer between the coupling device 100 and the mixing jar 150.

Referring again to FIGS. 1A-1D, the male fitting 110 of the coupling device 100 is formed by a cylindrical member 112 extending up from a central portion of the upwardly tapered top wall 122 of the female fitting 120. A standard thread 116 is provided on an outer surface 114 of the cylindrical member 112.

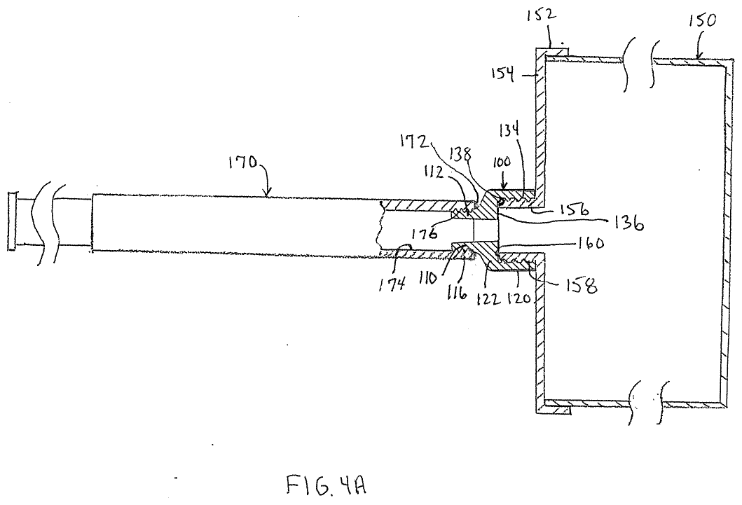

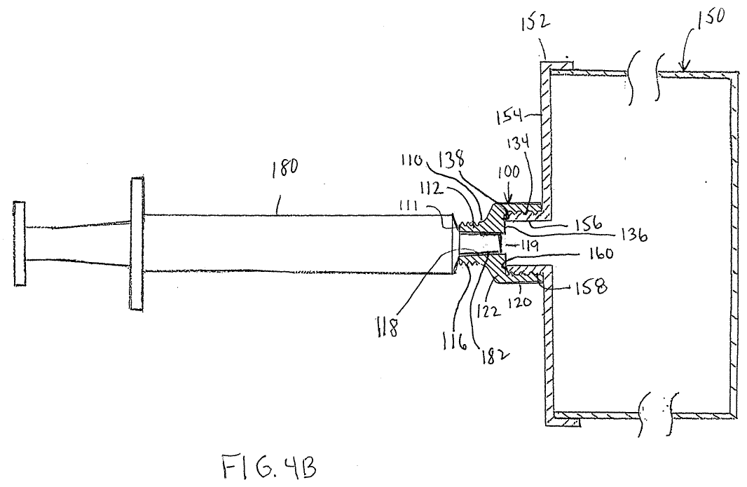

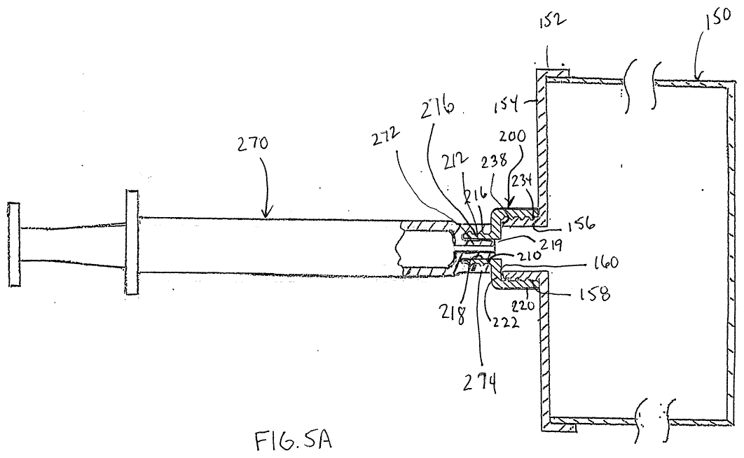

FIG. 4A shows the coupling device 100 coupled with a vaginal applicator 170. As shown, the thread 116 on the outer surface 114 of the male fitting 110 is adapted to threadedly engage a thread 176 provided on an inner surface 174 and at an outlet end 172 of a standard vaginal applicator 170. The threads 116, 176 operate to seal the vaginal applicator 170 to the coupling device 100 to prevent leakage during product transfer between the coupling device 100 and the applicator 170. In addition, the outlet end 172 of the vaginal applicator 170 engages the outer surface 114 of the male fitting when the applicator 170 is fully threaded and tightened to the coupling device 100, thereby providing a further seal to prevent product leakage.

Referring again to FIG. 1D, the cylindrical member 112 has an inner surface 118 that defines a tapered passageway 119, which extends through the cylindrical member 112 of the male fitting 110 and the top wall 122 of the female fitting 120 to allow the male fitting 110 to fluidly communicate with an interior 140 of the female fitting 120. The tapered passageway 119 tapers down in diameter moving from an outlet end 111 of the male fitting 110 towards the female fitting 120 to sealingly engage a tapered outlet end of an oral syringe thereby providing a leak resistant seal and retention of the syringe. The tapered passageway 119 has a taper of which can range from 1.00 degree to 20.30 degrees. In one preferred embodiment, the tapered passageway 119 has a taper of 5.03 degrees.

FIG. 4B shows the coupling device 100 coupled with an oral syringe 180. As shown, the tapered passageway 119 of the cylindrical member 112 of the male fitting 110 sealingly engages a tapered outlet end 182 of the oral syringe 280 to prevent leakage during product transfer between the coupling device 100 and the oral syringe 180.

The coupling device 100 of FIGS. 1A-1D can be made of plastic and formed as a unitary one-piece member.

The coupling device 100 of FIGS. 1A-1D ensures consistent filling volume and prevents over-tightening and provides the following advantages over current methods for both filling and dispensing/dosing from vaginal applicators:
Allows the compounding pharmacist to simply deliver the mixing jar, the newly invented coupling (which the pharmacist will screw onto the mixing jar with the appropriate standard industry cap included to seal it) and the vaginal applicators to the patient, eliminating the wasted product created by transferring to pumps, click dispensers or collapsible tubes.
By enabling the patient to fill easily and accurately their vaginal applicators, the pharmacist no longer has to fill collapsible tubes, or specialized pumps or click dispensers, saving the pharmacist tremendous amounts of time.
Eliminates the residual, wasted cream that cannot be recovered by the patient from pumps, click dispensers and collapsible tubes.
Provides a positive, consistent, and accurate connection point for the patient to dose and fill their applicators.
Reduces total cost by eliminating the need for pumps, click dispensers or collapsible tubes.
Unlike pumps/click dispensers, allows the prescribing doctor flexibility in changing dosages on the fly, using product already in the patient's possession.

In addition, the coupling device 100 of FIGS. 1A-1D provides connection of the mixing jar 150 to an outlet end of the oral syringe, which provides the following advantages over current methods for both filling and dispensing/dosing from oral syringes:
Allows the compounding pharmacist to simply deliver the mixing jar, the newly invented coupling (which the pharmacist will screw onto the mixing jar with the appropriate standard industry cap included to seal it) and the oral syringes to the patient, eliminating wasted product created by transferring to pumps/click dispensers.
Provides a snug, sealed fit so that the pharmacist or patient can easily fill oral syringes with no leaks, waste or mess.
By enabling the patient to accurately and effectively fill his/her own oral syringes, saves a significant amount of pharmacist preparation and packaging time.
Eliminates the residual, wasted cream that cannot be recovered by the patient from pumps/click dispensers.
Provides a positive, consistent, and accurate connection point for the patient to dose and fill their syringes.
Reduces total cost by eliminating the need for pumps/click dispensers.
Unlike pumps/click dispensers, allows the prescribing doctor flexibility in changing dosages on the fly, using product already in the patient's possession.
Eliminates the need for a separate adapter to connect the mixing jar tapered nozzle to oral syringes. The tapered nozzle-to-adapter-to syringe connections are a cumbersome assembly for the pharmacist or patient with the following problems associated with this method of filling oral syringes: non-positive, inaccurate fit which can result in wasted material and/or introduction of air making dosing inaccurate, significant difficulty for the patient to “manipulate” the syringe to achieve an accurate dose, and an increase in the time required to fill a syringe for either the pharmacist or the patient.
Serves two purposes with one coupling: connecting the mixing jar to both vaginal applicators and to oral syringes.

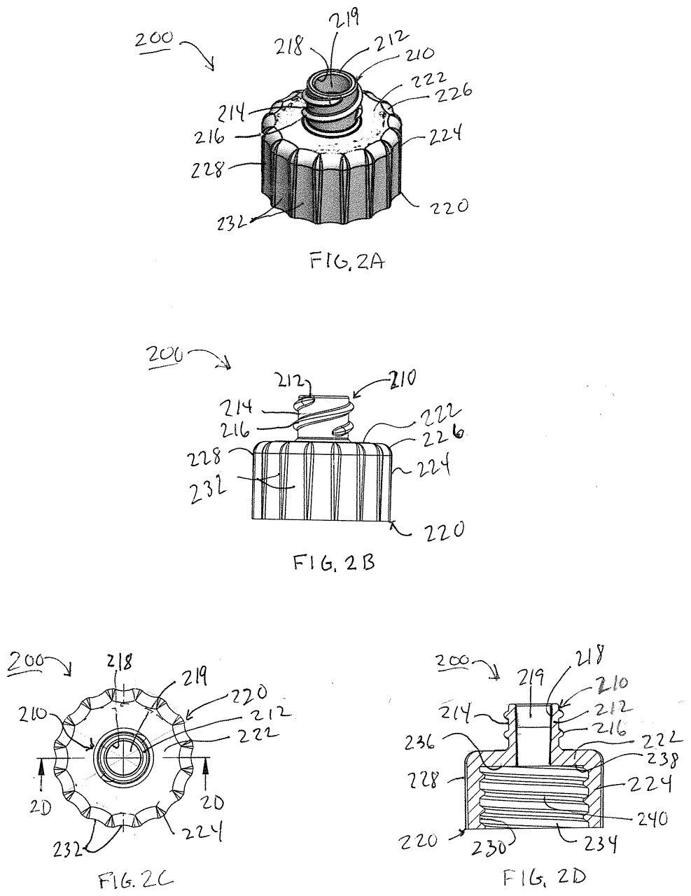

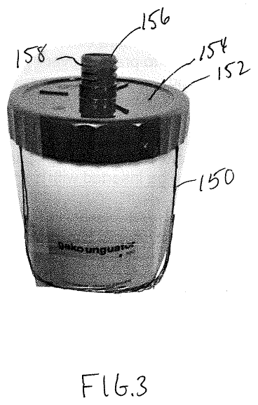

Referring now to FIGS. 2A-2D, there is shown a second embodiment of a coupling device 200 for coupling a mixing jar (e.g., EMP jar) to a Luer-Lock syringe or a Slip-Tip syringe. The coupling device 200 generally comprises a male fitting 210 and a female fitting 220.

The female fitting 220 of the coupling device 200 comprises a planar circular top wall 222 and a cylindrical side wall 224 depending from a rounded peripheral edge 226 of the top wall 222. The cylindrical side wall 224 of the female fitting 220 has an outer surface 228 and an inner surface 230. The outer surface 228 of the side wall 224 includes a plurality of longitudinal scallops or grooves 232 which extend into the rounded peripheral edge 226 of the top wall 222 and aid in gripping the coupling device 200 when attaching a Luer-Lock or Slip-Tip syringe to the coupling device 200 and/or when connecting the coupling device 200 to the mixing jar 150 (FIG. 3).

As shown FIGS. 2D, 3, 5A and 5B, the standard female or internal thread 234 is provided on the inner surface 230 of the cylindrical side wall 224. The female or internal thread 234 of the female fitting 220 is adapted to complement the male thread 158 provided on the male fitting 156 on the lid 152 of the mixing jar 150 (FIG. 3). The internally threaded female fitting 220 allows the coupling device 200 to be screw-threaded onto the lid 152 of the mixing jar 150. An inner surface 236 of the top wall 222 of the female fitting 220 defines a shoulder 238 above the internal thread 234 which is operative as a stop to ensure exact screw-tightening height and to prevent overtightening of the female fitting 220 of the coupling device 200 to the male fitting 156 of the mixing jar lid 152 (FIG. 3). More particularly, the free end 160 of the male fitting 156 of the mixing jar lid 152 will abut against the shoulder 238 of the female fitting 220 when the coupling device 200 is properly screw-tighten to the male fitting 156 of the mixing jar lid 152. The stop 238 is also operative as a seal to prevent leakage during product transfer between the coupling device 200 and the mixing jar 150.

Referring again to FIGS. 2A-2D, the male fitting 210 of the coupling device 200 is formed by a cylindrical member 212 extending up from the top wall 222 of the female fitting 220 and a standard Luer-Lock thread 216 provided on an outer surface 214 of the cylindrical member 212. The cylindrical member 212 has an inner surface 218 that defines a tapered passageway 219, which extends through the cylindrical member 212 and the top wall 222 of the female fitting 220 to allow the male fitting 210 to fluidly communicate with an interior 240 of the female fitting 220. The tapered passageway 219 tapers down in diameter moving from an outlet end of the male fitting 210 towards the female fitting 220 to sealingly engage a tapered outlet end of a Luer-Lock or Slip-Tip syringe, thereby providing a leak resistant seal and retention of the syringe. The taper of the tapered passageway 219 ranges from 1.00 degree to 10.72 degrees. In one preferred embodiment, the tapered passageway 219 has a taper of 2.86 degrees.

As in the previous embodiment, the coupling device 200 can be made of plastic and formed as a unitary one-piece member.

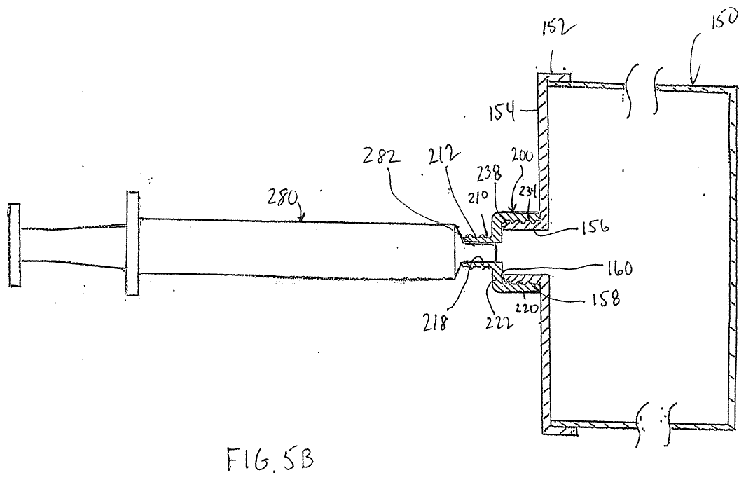

As shown in FIG. 5A, the Luer-Lock thread 216 of the male fitting 210 of the coupling device 200 allows a standard a Luer-Lock syringe 270 to be attached to the coupling device 200 by threadedly engaging the thread 216 of the male fitting 210 with an internal thread 276 of a female Luer-Lock fitting 274, which surrounds a tapered dispensing tip 272 of the Luer-Lock syringe 270. As the syringe 270 is threadedly engaged with the male fitting 210 of the coupling device 200, the tapered dispensing tip 272 of the syringe 270 enters and sealingly engages the tapered passageway 219 of the male fitting 210 to prevent leakage during product transfer between the coupling device 200 and the Luer-Lock syringe 270. Once fully threaded, the free end of the syringe's female Luer-lock fitting 274 engages the planar top wall 222 of the female fitting 220 of the device 200 to further insure against product leakage during product transfer between the coupling device 200 and the Luer-Lock syringe 270.

FIG. 5B shows the coupling device 200 coupled with a Slip-Tip syringe 280. As shown, the tapered passageway 219 of the cylindrical member 212 of the male fitting 210 sealingly engages a tapered outlet end 282 of the Slip-Tip syringe 280 to prevent leakage during product transfer between the coupling device 200 and the Slip-Tip syringe 280.

The coupling device 200 of FIGS. 2A-2D provides the following advantages over current methods for both filling and dispensing/dosing from Luer-Lock syringes:
Provides pharmacists/patients with a more efficient and accurate way to fill Luer-Lock syringes directly from a mixing jar using a positive connection.
Provides a snug, sealed fit so that the pharmacist or patient can easily fill Luer-Lock syringes with no leaks, waste or mess.
Eliminates the need for a separate adapter to connect the mixing jar tapered nozzle to Luer-Lock syringes. The tapered nozzle-to-adapter-to syringe connections are a cumbersome assembly for the pharmacist or patient with the following problems associated with this method of filling Luer-Lock syringes: non-positive, inaccurate fit which can result in wasted material and/or introduction of air making dosing inaccurate, significant difficulty for the patient to “manipulate” the syringe to achieve an accurate dose, and an increase in the time required to fill a syringe for either the pharmacist or the patient.

In addition, because the coupling device 200 of FIGS. 2A-2D is constructed to provide for the connection of a mixing jar to the top of a Slip-Tip syringe, the following advantages are realized over current methods for both filling and dispensing/dosing from Slip-Tip syringes:
Allows the compounding pharmacist to simply deliver the mixing jar, the coupling (which the pharmacist will screw onto the mixing jar with the appropriate standard industry cap included to seal it) and the appropriate Slip-Tip syringes to the patient, eliminating the wasted product created when transferring to pumps/click dispensers.
Provides a snug, sealed fit so that the pharmacist or patient can easily fill Slip-Tip syringes with no leaks, waste or mess.
By enabling the patient to accurately and effectively fill his/her own slip-tip syringes, saves a significant amount of pharmacist preparation and packaging time.
Eliminates the residual, wasted cream that cannot be recovered by the patient from pumps/click dispensers.
Provides a positive, consistent, and accurate connection point for the patient to dose and fill their Slip-Tip syringes.
Reduces total cost by eliminating the need for pumps/click dispensers.
Unlike pumps click dispensers, allows the prescribing doctor flexibility in changing dosages on the fly, using product already in the patient's possession.
Eliminates the need for a separate adapter to connect the mixing jar tapered nozzle to Slip-Tip syringes. The tapered nozzle-to-adapter-to syringe connections are a cumbersome assembly for the pharmacist or patient with the following problems associated with this method of filling slip-tip syringes: non-positive, inaccurate fit which can result in wasted material and/or introduction of air making dosing inaccurate, significant difficulty for the patient to “manipulate” the syringe to achieve an accurate dose, and an increase in the time required to fill a syringe for either the pharmacist or the patient.
Serves two purposes with one coupling: connecting the mixing jar to both Luer-Lock and Slip-Tip type syringes.

It should be understood that the invention is not limited to the embodiments illustrated and described herein. Rather, the appended claims should be construed broadly to include other variants and embodiments of the invention, which may be made by those skilled in the art without departing from the scope and range of equivalents of the invention. It is indeed intended that the scope of the invention should be determined by proper interpretation and construction of the appended claims and their legal equivalents, as understood by those of skill in the art relying upon the disclosure in this specification and the attached drawings.

What is claimed is:

1. A coupling device comprising:
a female fitting adapted attach a mixing jar thereto; and
a male fitting adapted to attach a vaginal applicator or an oral syringe thereto; or
a male fitting adapted to attach a Luer-Lock syringe or a Slip-Tip syringe thereto.

2. The coupling device of claim 1, wherein the female fitting includes a top wall and a cylindrical wall depending from a peripheral edge of the top wall.

3. The coupling device of claim 2, wherein the cylindrical wall includes an inner surface and a thread disposed on the inner surface.

4. The coupling device of claim 1, wherein the male fitting includes a cylindrical member disposed on the top wall of the female fitting.

5. The coupling device of claim 4, wherein the cylindrical member has an outer surface, an inner surface, a thread disposed on the outer surface and the inner surface defining a passageway.

6. The coupling device of claim 5, wherein the thread is a screw-thread or a Luer-Lock thread.

7. The coupling device of claim 6, wherein the thread is a screw-thread and wherein the inner surface is tapered.

8. A method for coupling a mixing jar to a vaginal applicator or an oral syringe, the method comprising:
providing a coupling device comprising:
a female fitting adapted to attach the mixing jar; and
a male fitting adapted to attach the vaginal applicator or the oral syringe thereto;
attaching the female fitting of the coupling device to the mixing jar; and
attaching the male fitting of the coupling device to the vaginal applicator or the oral syringe.

9. The method of claim 8, wherein the female fitting includes a top wall and a cylindrical wall depending from a peripheral edge of the top wall.

10. The method of claim 9, wherein the cylindrical wall includes an inner surface and a thread disposed on the inner surface.

11. The method of claim 9, wherein the male fitting includes a cylindrical member disposed on the top wall of the female fitting.

12. The method of claim 11, wherein the cylindrical member has an outer surface, an inner surface, a thread disposed on the outer surface and the inner surface defining a passageway.

13. The method of claim 12, wherein the inner surface is tapered.

14. A method for coupling a mixing jar to a Luer-Lock syringe or a Slip-Tip syringe, the method comprising:
providing a coupling device comprising:
a female fitting adapted to attach the mixing jar; and
a male fitting adapted to attach the Luer-Lock syringe or the Slip-Tip syringe thereto;
attaching the female fitting of the coupling device to the mixing jar; and
attaching the male fitting of the coupling device to the Luer-Lock syringe or the Slip-Tip syringe.

15. The method of claim 14, wherein the female fitting includes a top wall and a cylindrical wall depending from a peripheral edge of the top wall.

16. The method of claim 15, wherein the cylindrical wall includes an inner surface and a thread disposed on the inner surface.

17. The method of claim 14, wherein the male fitting includes a cylindrical member disposed on the top wall of the female fitting.

18. The method of claim 17, wherein the cylindrical member has an outer surface, an inner surface, a thread disposed on the outer surface and the inner surface defining a passageway.

19. The method of claim 18, wherein the thread is a Luer-Lock thread.

20. The method of claim 18, wherein the inner surface is tapered.
